(19) United States
(12) Patent Application Publication
(10) Pub. No.: US 2014/0329912 A1
(43) Pub. Date: Nov. 6, 2014

(54) CONTRACEPTIVE COMPOSITIONS AND METHODS OF CONTRACEPTION

(71) Applicant: James A. Barron, Belton, TX (US)

(72) Inventor: James A. Barron, Belton, TX (US)

(73) Assignee: INTEGRITY PHARMACEUTICALS, LLC, Temple, TX (US)

(21) Appl. No.: 14/348,710

(22) PCT Filed: Jul. 20, 2012
(86) PCT No.: PCT/US2012/047642

§ 371 Date: Jul. 17, 2014

Related U.S. Application Data
(60) Provisional application No. 61/541,250, filed on Sep. 30, 2011.

(51) Int. Cl. A61K 31/02

(52) U.S. Cl. CPC A61K 31/02
USPC 514/743

(57) ABSTRACT

Compositions and methods for contraception are disclosed that use 1-bromopropane.

### REFERENCE TO EARLIER FILED APPLICATION

This application claims the benefit under 35 U.S.C. §119(e) of U.S. Provisional Patent Application No. 61/541,250, filed Sep. 30, 2011, and titled “CONTRACEPTIVE COMPOSITIONS AND METHODS OF CONTRACEPTION,” which is incorporated, in its entirety, by this reference.

### TECHNICAL FIELD

This invention relates generally to methods of contraception and specifically to contraceptives using 1-bromopropane.

### BACKGROUND

Various procedures are known for controlling conception, and among the most reliable are surgical procedures such as tubal ligature for women and vasectomy for men. A problem with these techniques, however, is their permanence, i.e. irreversibility, although limited progress is being made in reversing vasectomies. For these reasons, chemical means are more widely used and the most common chemicals are taken orally by women to prevent ovulation. This has gained wide acceptance but still has certain drawbacks. Insofar as the male is concerned, he cannot be certain that the female has appropriately taken a contraceptive medication.

In addition, contraceptive medication taken by females often produces undesirable side effects, such as formation of blood clots. In such cases, it may be inadvisable to utilize an alternate technique for contraception. A chemical or chemically enhanced contraceptive for the male mate may be desirable.

### SUMMARY

In one aspect, a pharmaceutical composition is disclosed which includes (a) 1-bromopropane and (b) a pharmaceutically acceptable carrier.

In another aspect, a method of preventing contraception is disclosed by providing 1-bromopropane. In some embodiments, the method of preventing contraception includes providing a pharmaceutical composition that includes 1-bromopropane and a pharmaceutically acceptable carrier.

In another aspect, a method of preventing a male from impregnating a female is disclosed which includes administering a pharmaceutical composition that includes (a) 1-bromopropane and (b) a pharmaceutically acceptable carrier.

In another aspect, a method of preventing a male from impregnating a female is disclosed which includes administering a contraceptively effective amount of 1-bromopropane.

In another aspect, a method of contraception is disclosed which includes administering to the vagina an amount of a composition containing 1-bromopropane sufficient to render male sperm incapable of fertilization. In some embodiments, the 1-bromopropane is dispersed in a biocompatible, non-toxic vehicle.

In another aspect, a method for effecting contraception is disclosed which includes administering to a fertile mammalian male an effective contraceptive amount of 1-bromopropane. In some embodiments, a pharmaceutical composition comprising a pharmaceutically acceptable carrier is included.

### BRIEF DESCRIPTION OF THE DRAWINGS

FIG. 1 shows data from motility experiments described in the examples section.

### DETAILED DESCRIPTION

While the terminology used in this application is standard within the art, the following definitions of certain terms are provided to assure clarity.

Units, prefixes, and symbols may be denoted in their SI accepted form. Numeric ranges recited herein are inclusive of the numbers defining the range and include and are supportive of each integer within the defined range. Unless otherwise noted, the terms “a” or “an” are to be construed as meaning “at least one of.” The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any permissible purpose.

As used herein, the term “therapeutically effective amount” refers to an amount of 1-bromopropane (also sometimes referred to as n-propyl bromide) which is effective in reducing the fertility of a male. 1-Bromopropane is, therefore, useful in reducing male fertility.

As used herein, a “patient” refers to one in need of treatment for a disease or condition affected by reducing male fertility. The identification of those patients who are in need of treatment for the conditions identified herein is well within the ability and knowledge of one skilled in the art. A clinician skilled in the art can readily identify, by the use of clinical tests, physical examination and medical/family history, those patients who are in need of such treatment. A patient includes a warm-blooded animal such as a mammal which is in need of modulated protein kinase activity. It is understood that guinea pigs, dogs, cats, rats, mice, horses, cattle, sheep, and humans are examples of animals within the scope of the meaning of the term.

1-Bromopropane can be administered in any conventional form such as by inhalation, injection, drinking of a solution or by orally taking pills. For ease of storage, certainty of dosage and certainty of administration, pills may be used, and the dosage is adjusted so that the pill is taken in appropriate dosing regimen. For example, in some embodiments, 1-bromopropane may be dosed once daily. In other embodiments, 1-bromopropane may be dosed more than once per day.

In some embodiments, a therapeutically effective amount of n-propylbromide will be a concentration of from about 5 to about 100 ppm. In some embodiments, the concentration may be from about 20 to about 75 ppm. In some embodiments, the concentration may be from about 35 to about 65 ppm. In some embodiments, the concentration may be about 50 ppm. In some embodiments, doses of from about 0.0005 kg/l to about 0.01 kg/l may be taken. In some embodiments, doses of from about 0.1 to about 1000 mg/kg/day may be taken. In some embodiments, doses of from about 1 to 100 mg/kg/day may be taken. In some embodiments, doses of from about 5 to about 100 mg/kg/day may be taken. In some embodiments, doses of from about 20 to about 75 mg/kg/day may be taken. In some embodiments, doses of from about 35 to about 65 mg/kg/day may be taken. In some embodiments, doses totaling about 50 mg/kg/day may be taken.

In some embodiments, 1-bromopropane is diluted or compounded with pharmacologically acceptable carriers and/or diluents. For administration as a solution, the diluent may be water which may contain various flavoring agents, and the like. For administration by injection the diluent may be a saline solution and injection may be intravenous, subcutaneous, or the like. For pill form, the diluent or carrier may be lactose, glucose, starch, mannitol, magnesium stearate, microcrystalline cellulose, and the like. Other medicaments such as vitamins or the like may also be incorporated. The pills may be in the form of filled gelatin capsules, or can be compressed tablets or lozenges, in accordance with conventional pharmaceutical practice. Other adjuvants, such as lubricants or binding agents, for example, vegetable gums, or polyvinylpyrrolidone, may be incorporated into the dosage forms, if desired.

1-Bromopropane may be administered in any fluid or ointment vehicle suitable for topical or vaginal administration. Thus creams, ointments, foams, suppositories, ovules and the like may be formulated in which the 1-bromopropane is dispersed in a non-toxic vehicle suitable for topical and in particular for vaginal administration. Such vehicles include white petrolatum, hydrophilic petrolatum, lanolin emulsions, polyethylene glycols, cocoa butter and the like. Useful vehicles include emollient oils such as water-soluble oils, e.g., liquid polyethylene glycols, which promote complete and uniform distribution of the medicament within the vagina. Representative suitable vehicles include a lubricating jelly comprised of water, propylene glycol, hydroxyethyl cellulose, benzoic acid and sodium hydroxide, a water-soluble oil comprised of water, glycerin, propylene glycol, polyquaternium #5, methyl paraben and propyl paraben; a cream comprised of benzyl alcohol, cetearyl alcohol, cetyl esters wax, octyldodecanol, polysorbate 60, purified water, and sorbitan monostearate; and a suppository comprised of polyethylene glycol (PEG) 18, PEG-32, PEG-20 stearate, benzethonium chloride, methyl paraben and lactic acid.

In one aspect, a dispersion, suspension, or solution of 1-bromopropane in the vehicle may be introduced into the vagina in order to prevent conception during sexual intercourse. The amount of 1-bromopropane to be applied will be an amount that is effective to prevent conception or substantially reduce the risk thereof.

In another aspect, 1-bromopropane may be administered into a device which will remain in the vagina and dispense 1-bromopropane over a period of time in order to maintain an effective concentration in the vagina. Such a device may also be designed to provide a barrier that will prevent the access of the male sperm into the uterus and may also function itself as a contraceptive device.

In another aspect, 1-bromopropane may be administered into a device that encloses the penis and dispenses 1-bromopropane over a period of time in order to maintain an effective concentration in the device. Such a device may also be designed to provide a barrier that will prevent the access of the male sperm into the uterus and may also function itself as a contraceptive device. In one example, the device is a condom.

It is an advantage of using 1-bromopropane in a contraceptive composition that, after an initial period of taking the active material, contraception is effected but, while reversible, there is a delay or recovery period so that failure to take the medication one day will not contravene the conceptive incapacity of the male even for that day.

The safety and effectiveness of the active materials are shown in the following examples.

### EXAMPLES

Sperm Extraction from Mice

Male BALB/c mice (12-16 week old) were euthanized by CO₂-inhalation. The cauda epididymes was clipped and the upper portions of the vas deferens freed from surrounding tissue. Three-four cuts were made using scissors and forceps across the epididymes and several cuts along the length of the vas deferens. Sperm were allowed to swim out of the tissue. The dish was placed in a 5% CO₂ incubator set at 37° C. for 10-15 minutes. The solution of sperm was diluted (50 μl sperm+450 μl complete medium) in Whittens/HCO₃/BSA. Sperm was removed from the very top or sides of sperm drop to obtain the sperm with highest motility. The dish was then returned to the 5% CO₂ incubator set at 37° C. to allow the sperm to capacitate for 1-3 hrs prior to use.

### Motility Assay

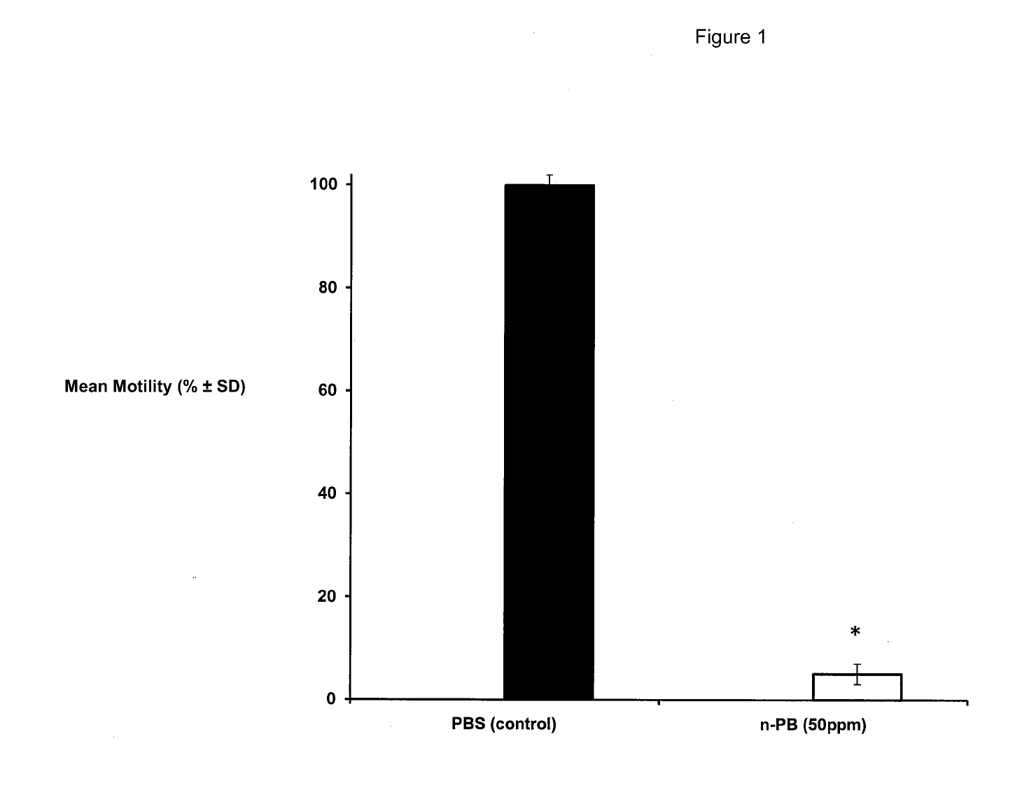

Sperms (10⁴) were placed in 96-well plate in complete media and treated with n-propyl bromide (n-PB) (50 ppm) or phosphate buffered saline (PBS) (control) and incubated for 10 minutes in a 5% CO₂ incubator set at 37° C. The number of motile sperm were counted in 4 wells and the mean recorded. The observed data is shown in FIG. 1.

### Real-Time PCR for Disease Pathways Finder

Sperms (10⁴) were placed in 96-well plate in complete media and treated with n-propyl bromide (50 ppm) or phosphate buffered saline (control) and incubated for 10 minutes in a 5% CO₂ incubator set at 37° C. The dish with sperm was then washed 3-times with phosphate buffered saline and returned to the 5% CO₂ incubator set at 37° C. for a further 24 hours. Sperm were then collected by centrifugation and subjected to the Stress and Toxicity PathwayFinder™ RT² Profiler™ assay according to the manufacturer's instructions (SA Biosciences, Frederick, Md.). This PCR Array profiles the expression of 84 genes whose expression level is indicative of stress and toxicity. The array includes genes that are directly up-regulated by oxidative or metabolic stress and by heat shock. The array also includes genes representative of pathways activated by prolonged stress, which range from cell death by apoptosis or necrosis to growth arrest and senescence to proliferation and carcinogenesis. The data collected from those experiments is shown in Table I.

TABLE I

| | | | Fold Change | |
|---|---|---|---|---|
| | | | PBS | n- |
| Gene | | | (con- | propyl |
| Symbol | Ref. Seq. | Gene Name | trol) | bromide |

Cell Cycle Control and DNA Damage Repair

| | | | | |
|---|---|---|---|---|
| ATM | NM_00051 | Ataxia telangiectasia | 1.33 | −1.01 |
| | | mutated | | |
| BRCA1 | NM_007294 | Breast cancer1, early onset | −1.09 | −1.23 |
| CCNE1 | NM_001238 | Cyclin E1 | −1.46 | −1.06 |
| CDC25A | NM_001789 | Cell division cycle 25, | −1.20 | −1.04 |
| | | homolog A | | |
| CDK4 | NM_000075 | Cyclin-dependent kinase 4 | −1.13 | −1.04 |
| CDKNA1 | NM_000389 | Cyclin dependent kinase | −1.06 | −1.25 |
| | | inhibitor 1A | | |
| CDKN2A | NM_000077 | Cyclin dependent kinase | −1.18 | −1.05 |
| | | inhibitor 2A | | |
| CHEK2 | NM_007194 | CHk2, Checkpoint homolog | −1.00 | −1.02 |
| E2F1 | NM_005225 | E2F transcription factor 1 | −1.28 | −1.17 |
| MDM2 | NM_002392 | MDM2 p53 binding protein | −1.04 | −1.32 |
| | | homolog | | |
| RB1 | NM_000321 | Retinoblastoma 1 | −1.15 | −1.30 |
| S100A4 | NM_002961 | S100 calcium binding | −1.10 | −1.15 |
| | | protein A4 | | |
| TP53 | NM_000546 | Tumor protein p53 | −1.04 | −1.06 |

Apoptosis and Senescence

| | | | | |
|---|---|---|---|---|
| APFA1 | NM_001160 | Apoptotic peptidase | −1.15 | −1.04 |
| | | activating factor 1 | | |
| BAD | NM_004322 | BCL2 associated agonist of | −1.16 | −1.00 |
| | | cell death | | |
| BAX | NM_004324 | BCL2 associated X protein | −1.05 | −1.25 |
| BCL2 | NM_000633 | B-cell CLL/lymphoma 2 | −1.18 | −1.24 |
| BCL2L1 | NM_138578 | BCL2-like 1 | −1.37 | −1.06 |
| CASP8 | NM_001228 | Caspase 8, apoptosis related | −1.03 | −1.57 |
| | | cysteine peptidase | | |
| CFLAR | NM_003879 | CASP8 & FADD like | −1.19 | −1.11 |
| | | apoptosis regulator | | |
| GZMA | NM_006144 | Granzyme A | −1.18 | −1.26 |
| HTATIP2 | NM_006410 | HIV-1 tat interactive | −1.06 | −1.38 |
| | | protein 2 | | |
| TERT | NM_198253 | Telomerase reverse | −1.15 | −1.16 |
| | | transcriptase | | |
| TNFRS1A | NM_001065 | Tumor necrosis factor | −1.01 | −1.36 |
| | | receptor super family | | |
| | | member 1A | | |
| TNFRSF10B | NM_003842 | Tumor necrosis factor | −1.08 | −1.33 |
| | | receptor super family | | |
| | | member 10b | | |
| TNFRSF25 | NM_003790 | Tumor necrosis factor | −1.06 | −1.15 |
| | | receptor super family | | |
| | | member 25 | | |

The data in Table I shows that no significant cell death, necrosis, mutigencity, or DNA damage was observed in the assays using n-propyl bromide where a two-fold or greater change might be significant of such an indication.

### Toxicity Screening

In addition, cell viability with n-propyl bromide was measured by MTS assay (Promega, Madison, Wis.). MTS [3-(4,5-dimethylthiazol-2-yl)-5-(3-carboxymethoxyphenyl)-2-(4-sulfophenyl)-2H-tetrazolium, inner salt] was utilized according to the manufacturer's instructions. The culture growth medium consisted of Whittens-HEPES complete media, (pH 7.2-7.4) prepared and supplemented with 30 mM NaHCO₃ and 10 mg/mL BSA (Bovine Serum Albumin, Sigma A0281, fatty acid free). 250 mL of Whittens-Hepes complete media includes 1461 mg of NaCl, 87.6 mg of KCl, 40.8 mg of KH₂PO₄, 36.1 mg of MgSO₄, 247.8 mg of glucose (dextrose), 22.01 mg of pyruvic acid, 130.92 mg of lactic acid (hemi-Ca), and 1191.5 mg of HEPES. After treatments of the sperms, 30 μL of MTS solution was added to each well and pipetted up and down for ten times to ensure properly mixing and incubation was prolonged for 2 h at 37° C., 5% CO₂.

Results of the MTS assay are shown in Table II. Absorbance observations for trials 1-3 and calculated average are expressed as optical density. The absorbance of cell-reduced MTS was read at 490 nm on a spectrophotometer Spectra Max Plus 384, and optical density for absorbance readings is provided in Table II. Absorbance is directly proportional to the number of living cells in culture. Thus, a reduction in absorbance normalized to a control indicates toxicity to a cell. Background absorbance was measured for the culture media only in three trials. An average absorbance was calculated from the three trials. The average optical density for the three trials for the culture media (background) was then subtracted from the average optical density of the various concentration assays for n-propyl bromide (expressed in terms of ppm of n-propyl bromide). Three control trials using only phosphate buffered saline were also measured. The corrected, average optical density of each of the experimental assays was then compared to the corrected, average optical density of the control (PBS only) assays. The comparison is expressed as the percentage of MTS reduction relative to the normalized absorbance of control samples. The data in Table II reflects that at concentrations of 50 ppm or less, n-propyl bromide does not result in significant cell toxicity.

TABLE II

| | Culture | | | | | | | | | | PBS |
|---|---|---|---|---|---|---|---|---|---|---|---|
| | Media | 600 ppm | 200 ppm | 100 ppm | 50 ppm | 25 ppm | 12.5 ppm | 5 ppm | 1 ppm | 0.1 ppm | only |
| Trial 1 | 0.2 | 1.2 | 0.8 | 0.4 | 0.2 | 0.3 | 0.4 | 0.2 | 0.2 | 0.2 | 0.3 |
| Trial 2 | 0.2 | 1.1 | 0.9 | 0.6 | 0.5 | 0.4 | 0.4 | 0.3 | 0.4 | 0.1 | 0.2 |
| Trial 3 | 0.1 | 0.9 | 0.7 | 0.7 | 0.1 | 0.2 | 0.1 | 0.4 | 0.1 | 0.3 | 0.3 |
| Average | 0.2 | 1.1 | 0.8 | 0.6 | 0.3 | 0.3 | 0.3 | 0.3 | 0.2 | 0.2 | 0.3 |
| Corrected | 0.0 | 0.9 | 0.6 | 0.4 | 0.1 | 0.1 | 0.1 | 0.1 | 0.1 | 0.0 | 0.1 |
| % of | | 900 | 600 | 400 | 100 | 100 | 100 | 100 | 100 | 90 | 100 |
| control | | | | | | | | | | | |

It will be appreciated that the instant specification and examples are set forth by way of illustration and not limitation, and that various modifications and changes may be made without departing from the spirit and scope of the present disclosure.

1. A pharmaceutical composition comprising:
(a) 1-bromopropane; and
(b) a pharmaceutically acceptable carrier.

2. (canceled)

3. A method of preventing contraception comprising providing the pharmaceutical composition of claim 1.

4. (canceled)

5. (canceled)

6. A method of contraception comprising administering a prophylactically effective amount of 1-bromopropane sufficient to render male sperm incapable of fertilization.

7. The method of claim 6, wherein 1-bromopropane is dispersed in a biocompatible, non-toxic vehicle.

8. A method for affecting contraception comprising administering to a fertile mammal an effective contraceptive amount of 1-bromopropane.

9. The method of claim 8, wherein 1-bromopropane is administered in a pharmaceutical composition comprising a pharmaceutically acceptable carrier.

10. The method of claim 8, wherein the mammal is male.

11. The method of claim 8, wherein the mammal is female.

12. The method of claim 8, wherein the mammal is not human.

13. The method of claim 8, wherein the mammal is human.

14. The method of claim 9, wherein the mammal is human.

15. The method of claim 9, wherein the mammal is not human.

16. The method of claim 10, wherein the mammal is human.

17. The method of claim 10, wherein the mammal is not human.

18. The method of claim 11, wherein the mammal is human.

19. The method of claim 11, wherein the mammal is not human.

20. The method of claim 6, wherein the administration is to male semen.

21. The method of claim 6, wherein the administration is to a vagina.

22. The method of claim 20, wherein the semen is human semen.

23. The method of claim 21, wherein the semen is not human semen.
